(19) United States
(12) Patent Application Publication
(10) Pub. No.: US 2023/0115432 A1
(43) Pub. Date: Apr. 13, 2023

(54) Methods and Apparatuses for Early Diagnosis of Lung Infection Acuity

(71) Applicant: Diagnose Early, Inc., Newark, CA (US)

(72) Inventors: Chris Wheeler, Newark, CA (US); Karl-Magnus Larsson, Newark, CA (US); Jeffrey A. Schuster, Newark, CA (US)

(21) Appl. No.: 17/912,969

(22) PCT Filed: Mar. 26, 2021
(86) PCT No.: PCT/US2021/024347

§ 371 Date: Sep. 20, 2022

Related U.S. Application Data
(60) Provisional application No. 63/000,452, filed on Mar. 26, 2020.
(60) Provisional application No. 63/005,148, filed on Apr. 3, 2020.

(51) Int. Cl. A61B 5/08; A61B 5/00; C12Q 1/70; G01N 33/569

(52) U.S. Cl. CPC A61B 5/082; A61B 5/7275; C12Q 1/70; G01N 33/569; G01N 2800/12

(57) ABSTRACT

The present disclosure provides methods and devices for early diagnosis of an infection, for example by the COVID-19 virus, and determining a course of action for treatment of the infection.

### CROSS-REFERENCE TO RELATED PATENT APPLICATIONS

This patent application claims a benefit of priority to U.S. Provisional Patent Applications 63/000,452 filed 26 Mar. 2020, and 63/005,148 filed 3 Apr. 2020, each of which is incorporated by reference herein for all purposes.

### TECHNICAL FIELD

This disclosure relates to methods and apparatuses for early diagnosis of an infection, for example by a COVID-19 virus, and determining a course of action for treatment of the infection.

### BACKGROUND

In this disclosure, where a document, an act, and/or an item of knowledge is referred to and/or discussed, then such reference and/or discussion is not an admission that the document, the act, and/or the item of knowledge and/or any combination thereof was at a priority date, publicly available, known to a public, part of common general knowledge, and/or otherwise constitutes any prior art under any applicable statutory provisions; and/or is known to be relevant to any attempt to solve any problem with which this disclosure is concerned with. Further, nothing is disclaimed.

Some new human virus or bacteriological outbreaks can arise at any time. Some new viruses are often created by an acquisition of new variants in animals that allow a spread of these viruses in humans. Some examples of these viruses include COVID-19, SARS, Ebola, and influenza. If an extent of such spread is high, then a pandemic occurs. Some of these viruses are associated with lung infection. If a new pandemic arises, then diagnostic tests, pharmaceutical treatments, hospital beds and staff, medical supplies, such as respirators and endotracheal tubes, and medical equipment, such as ventilators, may be in short supply. Best outcomes can be achieved when steps are taken to limit a transmission of these viruses, thereby limiting a number of severe cases at any given time, and targeting intensive treatment toward those who are most likely to benefit.

Many infections, such as pandemic viral infections, have significant mortality and morbidity associated with lung infections (e.g., pneumonia). Some lung infections can cause an infectious agent, for example virus, to spread without direct contact via exhaled droplets.

Some corona viruses, such as SARS, can be associated with pulmonary fibrosis (Tse et al., J. Clin. Path. 57:260-265, 2004). Some chest CT scans of some patients with confirmed (via real time RT-PCR) COVID-19 show ground glass opacities suggestive of fibrosis development. (She et al., J. Med. Virol. 92:747-754, 2020). Some active and ongoing formation of lung scar tissue, i.e., progressing pulmonary fibrosis, can be associated with elevated levels of certain biomarkers, specifically proline, 4-hydroxyproline, alanine, valine, leucine/isoleucine and allysine (Gaugg et al., Respirology 24:437-444, 2019), which can also be found in analyzed lung tissue in pulmonary fibrosis (Kang et al., J. Proteome Res. 15:1717-1724, 2016). Currently, some 02 saturation and x-ray measurements of ground glass opacities can be used to diagnose some lung function and some scarring. However, neither of these methods, without expensive and time-consuming repetitions of these tests, can measure a rate of deterioration of lung functionality, which can indicate an acuity of certain viral infections and diseases, for example, COVID-19. Therefore, there is a technological need for method and apparatuses of measuring the rate of deterioration of lung functionality, for example, due to progressive lung scarring and pulmonary fibrosis, in order to detect the acuity of certain viral infections and diseases.

There is also a technological problem to be solved, where the technological problem is early identification of those patients who are most likely to have a viral infection or disease that results in progressing pulmonary fibrosis, using this information to determine a predetermined, recommended, or best course of treatment for these patients, thereby targeting limited resources toward this subpopulation of patients. Further, the technological problem thus also involves early identification of those patients who are least likely to have a viral infection or disease that results in progressing pulmonary fibrosis, and using this information to target these patients for limited or no treatment and eliminate such patients from more aggressive treatment options, freeing up these more aggressive options for those patients that are most likely to have a viral infection or disease that results in progressing pulmonary fibrosis.

### SUMMARY

Generally, this disclosure enables various technologies (e.g., methods, apparatuses) for early detection of patients with viral infections or diseases that are more or most likely to have a viral infection or disease that results in progressing pulmonary fibrosis. In certain aspects, these technologies involve an analysis of a sampled exhaled breath for a biomarker associated with of a pathogenesis of a disease, for example a pandemic disease, for an early detection of the biomarker correlated with a progression of pulmonary fibrosis associated with a viral infection, for example COVID-19 infection (e.g., Wuhan variant, UK variant, South Africa variant, NYC variant), thereby identifying those patients most in need of more aggressive treatment options. In certain aspects, these technologies enable ways of reducing a demand for medications, tests, hospital beds, staff, equipment, and supplies during a pandemic, especially during peak demand. Thus, in certain aspects, these technologies can provide additional diagnostic techniques to provide access to health services, equipment, and supplies to those most currently in need. Likewise, in certain aspects, there is a need for additional screening methods to direct limited testing kits to those most likely to be suffering from infection or high acuity infection during a pandemic, which can be addressed by these technologies. These technologies also address various needs to enable methods and systems for real-time point of care analysis of sampled exhaled breath for biomarkers associated with the pathogenesis of a disease, for example, a pandemic disease, for example, some biomarkers correlated with the progression of pulmonary fibrosis associated with a viral infection, for example COVID-19 infection.

In certain aspects, this disclosure enables a method of allocating a medical resource during a pandemic, the method comprising: a) screening (e.g., sensors, diagnostic kits, bodily fluids) a set of patients based on known symptoms; b) testing for infection due to a pathogen; c) capturing exhaled breath from the patients, where the pathogen is removed from the exhaled breath as captured; d) determining a level of a compound in the exhaled breath for each patient of the set of patients; and e) based on the determined levels, allocating (e.g., moving, attaching, inputting) a set of medical resources (e.g., equipment, ventilators, drugs) to the patients. In certain aspects, the pathogen is a virus is selected from a corona virus, an influenza virus, a syncytial virus, a parainfluenza virus, an adenovirus, a rhinovirus, a metapneumovirus, an enterovirus, or a variant thereof. In other aspects, the pathogen is a virus, and the compound is a biomarker for progression of pulmonary fibrosis. In some aspects, the virus is COVID-19 (e.g., Wuhan variant) or any variant thereof (e.g., UK, South Africa, NYC). In further aspects, the testing comprises a real-time RT-PCR test. In yet further aspects, the medical resources are a hospital bed, a bed in an intensive care unit, a ventilator, an endotracheal tube, a doctor, a nurse, an x-ray, a CAT scan, a chemical composition, a biological composition, a monoclonal body, or an anti-viral medicine. In certain aspects, the pathogen is selected from a virus and a bacteria, and wherein the pathogen is removed using a filter. In certain aspects, the filter is selected from a glass fiber filter, a polycarbonate membrane filter, and an activated charcoal filter. In certain aspects, the filter has an effective pore size of about 0.3 μm or less. In certain aspects, the effective pore size is about 0.1 μm or less. In certain aspects, the filter removes about 95% or more, 96% or more, 97% or more, 98% or more, 99% or more, or 99.9% or more of the pathogen from the breath sample. In certain aspects, the exhaled breath goes through, transits, travels, or moves through the filter prior to being captured. In certain aspects, the exhaled breath is forced through the filter after being captured (e.g., via gravity, positive or negative pressure). In certain aspects, the exhaled breath is captured in a breath capture system (e.g., a single use system), and the breath capture system is disposed of after forcing the breath sample through the filter. In certain aspects, the testing is done after the breath capture, and the pathogen that is captured by the filter is analyzed to diagnose the infection by the pathogen. In certain aspects, the biomarker is proline, 4-hydroxyproline, alanine, valine, leucine/isoleucine, allysine or any combination thereof. In certain aspects, the testing comprises measuring the level of proline, 4-hydroxyproline, alanine, valine, leucine/isoleucine, or allysine. In certain aspects, the testing comprises measuring a ratio of the level of two or more, or four or more, or all of proline, 4 hydroxyproline, alanine, valine, leucine/isoleucine, or allysine. In certain aspects, the testing comprises a real-time point of care (rt-POC) test. In certain aspects, the rt-POC test returns results in less than or about 1 hour, less than or about 10 minutes, or less than or about 1 minute. In certain aspects, the rt-POC test comprises a system comprised of: a) a breath chamber; b) a sensor; and c) a system for displaying the result of the test (e.g., a processor and a display in operative communication with the processor where the processor is programmed accordingly); or d) a system for transmitting a result of the rt-POC test. In certain aspects, the system further comprises one or more of a durable component configured to attach to the breath chamber, a base unit, a biomarker detector, a reagent, a pump, a valve (e.g., a check valve, a ball valve, a butterfly valve), a reaction chamber, a filter, a display (e.g., LCD, electrophoretic, plasma, digital, analog), a flow rate sensor, a flow volume sensor, a pressure sensor, a system for calculating an exhalation flow rate, a system for calculating an exhaled volume, a display (e.g., LCD, electrophoretic, plasma, digital, analog), or a speaker. In certain aspects, the sensor is an electrochemical immunosensor, an optical immunosensor, a microgravimetric immunosensor, a thermometric immunosensor, an antibody immunosensor, an aptamer immunosensor, a microRNA immunosensor, meso-tetra (4-sulphonatophenyl) porphyrin (TPPS) immobilized onto a film, a colorimetric sensor, a photochromic sensor chip, an enzyme biosensor, an organic electrochemical transistors, a fluorescence sensor, an electrochemiluminescence sensor, a D-phenylalanine fluorescence sensor, an enantioselective sensor, a potentiometric sensor, an ODAP-selective sensor, an electrochemical metal ion sensor, a microcantilever sensor, or a supramolecular luminescent sensor.

In certain aspects, this disclosure also enables a method for testing for a biomarker associated with a progression of a pulmonary fibrosis, the method comprising: a) introducing a breath sample into a spirometry filter; b) diverting an amount of the breath sample into a carbon dioxide meter; c) heating the breath sample that is remaining to ionize the breath sample; d) introducing the breath sample as ionized into a sample inlet of a mass spectrometer; and e) detecting the biomarker associated with a progression of the pulmonary fibrosis. In certain aspects, the biomarker is proline, 4-hydroxyproline, alanine, valine, leucine/isoleucine, allysine or any combination thereof.

In certain aspects, this disclosure also enables a method of treating a patient for a viral or bacterial infection characterized by a progression of a pulmonary fibrosis, the method comprising: a) introducing a breath sample from a patient into a spirometry filter; b) ionizing the breath sample; c) introducing the breath sample as ionized into a sample inlet of a mass spectrometer; d) detecting a biomarker associated with the progression of the pulmonary fibrosis; and e) treating the patient with or based on the biomarker that was detected. In certain aspects, the patient has a viral infection. In certain aspects, the viral infection is a corona virus (e.g., COVID-19, Wuhan variant, UK variant, South Africa variant, NYC variant), an influenza virus, a syncytial virus, a parainfluenza virus, an adenovirus, a rhinovirus, a metapneumovirus and an enterovirus infection. In certain aspects, the biomarker is proline, 4-hydroxyproline, alanine, valine, leucine/isoleucine, allysine or any combination thereof.

### DESCRIPTION OF DRAWINGS

This disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a flowchart of one embodiment of this disclosure, in an embodiment wherein the test for the condition is fast and readily available.

FIG. 2 is a flowchart of another embodiment of this disclosure, wherein the test for the condition is in short supply and/or takes a long time to return results.

FIG. 3 is a flowchart of another embodiment of this disclosure, detailing the DECA test procedure separate from the clinical context, explaining the DECA text box as shown in FIG. 1 and FIG. 2.

FIG. 4 is a flowchart of another embodiment of this disclosure, wherein the physical process is detailed.

FIG. 5 shows one embodiment of a breath chamber for use in the presently disclosed devices and systems according to this disclosure.

FIG. 6 shows another embodiment of a breath chamber for use in the presently disclosed devices and systems according to this disclosure.

### DETAILED DESCRIPTION

Generally, this disclosure enables various technologies (e.g., methods, apparatuses) for early detection of patients with viral infections or diseases that are more or most likely to have a viral infection or disease that results in progressing pulmonary fibrosis. In certain aspects, these technologies involve an analysis of a sampled exhaled breath for a biomarker associated with of a pathogenesis of a disease, for example a pandemic disease, for an early detection of the biomarker correlated with a progression of pulmonary fibrosis associated with a viral infection, for example COVID-19 infection, thereby identifying those patients most in need of more aggressive treatment options. In certain aspects, these technologies enable ways of reducing a demand for medications, tests, hospital beds, staff, equipment, and supplies during a pandemic, especially during peak demand. Thus, in certain aspects, these technologies can provide additional diagnostic techniques to provide access to health services, equipment, and supplies to those most currently in need. Likewise, in certain aspects, there is a need for additional screening methods to direct limited testing kits to those most likely to be suffering from infection during a pandemic, which can be addressed by these technologies. These technologies also address various needs to enable methods and systems for real-time point of care analysis of sampled exhaled breath for biomarkers associated with the pathogenesis of a disease, for example, a pandemic disease, for example, some biomarkers correlated with the progression of pulmonary fibrosis associated with a viral infection, for example COVID-19 infection.

In certain aspects, this disclosure enables a method of allocating a medical resource during a pandemic, the method comprising: a) screening (e.g., sensors, diagnostic kits, bodily fluids) a set of patients based on known symptoms; b) testing for infection due to a pathogen; c) capturing exhaled breath from the patients, where the pathogen is removed from the exhaled breath as captured; d) determining a level of a compound in the exhaled breath for each patient of the set of patients; and e) based on the determined levels, allocating (e.g., moving, attaching, inputting) a set of medical resources (e.g., equipment, ventilators, drugs) to the patients. In certain aspects, the pathogen is a virus is selected from a corona virus, an influenza virus, a syncytial virus, a parainfluenza virus, an adenovirus, a rhinovirus, a metapneumovirus, an enterovirus, or a variant thereof. In other aspects, the pathogen is a virus, and the compound is a biomarker for progression of pulmonary fibrosis. In some aspects, the virus is COVID-19 (e.g., Wuhan variant) or any variant thereof (e.g., UK, South Africa, NYC). In further aspects, the testing comprises a real-time RT-PCR test. In yet further aspects, the medical resources are a hospital bed, a bed in an intensive care unit, a ventilator, an endotracheal tube, a doctor, a nurse, an x-ray, a CAT scan, a chemical composition, a biological composition, a monoclonal body, or an anti-viral medicine. In certain aspects, the pathogen is selected from a virus and/or a bacteria, and wherein the pathogen is removed using a filter. In certain aspects, the filter is selected from a glass fiber filter, a polycarbonate membrane filter, and an activated charcoal filter. In certain aspects, the filter has an effective pore size of about 0.3 μm or less. In certain aspects, the effective pore size is about 0.1 μm or less. In certain aspects, the filter removes about 95% or more, 96% or more, 97% or more, 98% or more, 99% or more, or 99.9% or more of the pathogen from the breath sample. In certain aspects, the exhaled breath goes through, transits, travels, or moves through the filter prior to being captured. In certain aspects, the exhaled breath is forced through the filter after being captured (e.g., via gravity, positive or negative pressure). In certain aspects, the exhaled breath is captured in a breath capture system (e.g., a single use system), and the breath capture system is disposed of after forcing the breath sample through the filter. In certain aspects, the testing is done after the breath capture, and the pathogen that is captured by the filter is analyzed to diagnose the infection by the pathogen. In certain aspects, the biomarker is proline, 4-hydroxyproline, alanine, valine, leucine/isoleucine, allysine or any combination thereof. In certain aspects, the testing comprises measuring the level of proline, 4-hydroxyproline, alanine, valine, leucine/isoleucine, or allysine. In certain aspects, the testing comprises measuring a ratio of the level of two or more, or four or more, or all of proline, 4 hydroxyproline, alanine, valine, leucine/isoleucine, or allysine. In certain aspects, the testing comprises a real-time point of care (rt-POC) test. In certain aspects, the rt-POC test returns results in less than or about 1 hour, less than or about 10 minutes, or less than or about 1 minute. In certain aspects, the rt-POC test comprises a system comprised of: a) a breath chamber; b) a sensor; and c) a system for displaying the result of the test (e.g., a processor and a display in operative communication with the processor where the processor is programmed accordingly); or d) a system for transmitting a result of the rt-POC test. In certain aspects, the system further comprises one or more of a durable component configured to attach to the breath chamber, a base unit, a biomarker detector, a reagent, a pump, a valve (e.g., a check valve, a ball valve, a butterfly valve), a reaction chamber, a filter, a display (e.g., LCD, electrophoretic, plasma, digital, analog), a flow rate sensor, a flow volume sensor, a pressure sensor, a system for calculating an exhalation flow rate, a system for calculating an exhaled volume, a display (e.g., LCD, electrophoretic, plasma, digital, analog), or a speaker. In certain aspects, the sensor is an electrochemical immunosensor, an optical immunosensor, a microgravimetric immunosensor, a thermometric immunosensor, an antibody immunosensor, an aptamer immunosensor, a microRNA immunosensor, meso-tetra (4-sulphonatophenyl) porphyrin (TPPS) immobilized onto a film, a colorimetric sensor, a photochromic sensor chip, an enzyme biosensor, an organic electrochemical transistors, a fluorescence sensor, an electrochemiluminescence sensor, a D-phenylalanine fluorescence sensor, an enantioselective sensor, a potentiometric sensor, an ODAP-selective sensor, an electrochemical metal ion sensor, a microcantilever sensor, or a supramolecular luminescent sensor.

In certain aspects, this disclosure also enables a method for testing for a biomarker associated with a progression of a pulmonary fibrosis, the method comprising: a) introducing a breath sample into a spirometry filter; b) diverting an amount of the breath sample into a carbon dioxide meter; c) heating the breath sample that is remaining to ionize the breath sample; d) introducing the breath sample as ionized into a sample inlet of a mass spectrometer; and e) detecting the biomarker associated with a progression of the pulmonary fibrosis. In certain aspects, the biomarker is proline, 4-hydroxyproline, alanine, valine, leucine/isoleucine, allysine or any combination thereof.

In certain aspects, this disclosure also enables a method of treating a patient for a viral or bacterial infection characterized by a progression of a pulmonary fibrosis, the method comprising: a) introducing a breath sample from a patient into a spirometry filter; b) ionizing the breath sample; c) introducing the breath sample as ionized into a sample inlet of a mass spectrometer; d) detecting a biomarker associated with the progression of the pulmonary fibrosis; and e) treating the patient with or based on the biomarker that was detected. In certain aspects, the patient has a viral infection. In certain aspects, the viral infection is a corona virus (e.g., COVID-19, Wuhan variant, UK variant, South Africa variant, NYC variant), an influenza virus, a syncytial virus, a parainfluenza virus, an adenovirus, a rhinovirus, a metapneumovirus and an enterovirus infection. In certain aspects, the biomarker is proline, 4-hydroxyproline, alanine, valine, leucine/isoleucine, allysine or any combination thereof.

The set of accompanying illustrative drawings shows various embodiments of this disclosure. Such drawings are not to be construed as necessarily limiting this disclosure. Like numbers and/or similar numbering scheme can refer to like and/or similar elements throughout.

This disclosure is now described more fully with reference to the set of accompanying drawings, in which some embodiments of this disclosure are shown. This disclosure may, however, be embodied in many different forms and should not be construed as necessarily being limited to the embodiments disclosed herein. Rather, these embodiments are provided so that this disclosure is thorough and complete, and fully conveys various concepts of this disclosure to skilled artisans.

Aspects of this disclosure are described with reference to flowchart illustrations and/or block diagrams of methods, apparatus (systems), and computer program products according to embodiments of the disclosure. Each block of the flowchart illustrations and/or block diagrams, and combinations of blocks in the flowchart illustrations and/or block diagrams, can be implemented by computer readable program instructions. The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The flowchart and block diagrams as included herewith illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Words such as “then,” “next,” etc. are not intended to limit the order of the steps; these words are simply used to guide the reader through the description of the methods. Although process flow diagrams may describe the operations as a sequential process, many of the operations can be performed in parallel or concurrently. In addition, the order of the operations may be re-arranged. A process may correspond to a method, a function, a procedure, a subroutine, a subprogram, etc. When a process corresponds to a function, its termination may correspond to a return of the function to the calling function or the main function.

Features or functionality described with respect to certain embodiments may be combined and sub-combined in and/or with various other embodiments. Also, different aspects and/or elements of embodiments, as disclosed herein, may be combined and sub-combined in a similar manner as well. Further, some embodiments, whether individually and/or collectively, may be components of a larger system, wherein other procedures may take precedence over and/or otherwise modify their application. Additionally, a number of steps may be required before, after, and/or concurrently with embodiments, as disclosed herein. Note that any and/or all methods and/or processes, at least as disclosed herein, can be at least partially performed via at least one entity or actor in any manner.

The terminology used herein can imply direct or indirect, full or partial, temporary or permanent, action or inaction. For example, when an element is referred to as being “on,” “connected” or “coupled” to another element, then the element can be directly on, connected or coupled to the other element and/or intervening elements can be present, including indirect and/or direct variants. In contrast, when an element is referred to as being “directly connected” or “directly coupled” to another element, there are no intervening elements present.

Although the terms first, second, etc. can be used herein to describe various elements, components, regions, layers and/or sections, these elements, components, regions, layers and/or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from the teachings of the present disclosure.

The terminology used herein is for describing particular embodiments and is not intended to be necessarily limiting of this disclosure. As used herein, the singular forms “a,” “an” and “the” are intended to include the plural forms (e.g., two, three, four, five, six, seven, eight, nine, ten, tens, hundreds, thousands, millions, or more) as well, including intermediate whole or decimal forms, unless the context clearly indicates otherwise. Also, as used herein, the term “a” and/or “an” shall mean “one or more,” even though the phrase “one or more” is also used herein. The terms “comprises,” “includes” and/or “comprising,” “including” when used in this specification, specify the presence of stated features, integers, steps, operations, elements, and/or components, but do not preclude the presence and/or addition of one or more other features, integers, steps, operations, elements, components, and/or groups thereof. Furthermore, when this disclosure states herein that something is “based on” something else, then such statement refers to a basis which may be based on one or more other things as well. In other words, unless expressly indicated otherwise, as used herein “based on” inclusively means “based at least in part on” or “based at least partially on.”

As used herein, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. For example, X includes A or B can mean X can include A, X can include B, and X can include A and B, unless specified otherwise or clear from context.

As used herein, the term “response” or “responsive” are intended to include a machine-sourced action or inaction, such as input (e.g., local, remote), or a user-sourced action or inaction, such as input (e.g., via user input device).

As used herein, the term “about” and/or “substantially” refers to a +/−10% variation from the nominal value/term. Such variation is always included in any given.

Unless otherwise defined, all terms (including technical and scientific terms) used herein have the same meaning as commonly understood by one of ordinary skill in the art to which this disclosure belongs. The terms, such as those defined in commonly used dictionaries, should be interpreted as having a meaning that is consistent with their meaning in the context of the relevant art and should not be interpreted in an idealized and/or overly formal sense unless expressly so defined herein.

Hereby, all issued patents, published patent applications, and non-patent publications that are mentioned or referred to in this disclosure are herein incorporated by reference in their entirety for all purposes, to a same extent as if each individual issued patent, published patent application, or non-patent publication were specifically and individually indicated to be incorporated by reference. To be even more clear, all incorporations by reference specifically include those incorporated publications as if those specific publications are copied and pasted herein, as if originally included in this disclosure for all purposes of this disclosure. Therefore, any reference to something being disclosed herein includes all subject matter incorporated by reference, as explained above. However, if any disclosures are incorporated herein by reference and such disclosures conflict in part or in whole with this disclosure, then to an extent of the conflict or broader disclosure or broader definition of terms, this disclosure controls. If such disclosures conflict in part or in whole with one another, then to an extent of conflict, the later-dated disclosure controls.

Before the present formulations and methods are described, it is to be understood that this disclosure is not limited to particular formulations and methods described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

The present disclosure relates to methods and devices for early diagnosis of an infection, for example by the COVID-19 virus (e.g., Wuhan variant, UK variant, South African variant, NYC variant), and determining a course of action for treatment of the infection. In certain embodiments, the method comprises: selecting a subject based on risk factors for significant mortality and morbidity due to a viral infection, checking for symptoms such as cough or fever, testing lung function, performing a diagnostic test for the infective agent, for example testing for COVID-19 using a Real-Time Reverse Transcriptase (RT)-PCR Diagnostic Panel, collecting exhaled breath in a way that excludes viruses and bacteria from the sample, analyzing the sample for evidence of biomarkers relating to expected acuity of active infection, such as biomarkers for progressive pulmonary fibrosis, and modifying treatment based on the results.

The present disclosure relates to methods and devices for determining the acuity of an infection, for example by COVID-19 virus. This disclosure enables a method of screening subjects who have tested positive for an infection, for example a COVID-19 infection, to determine the acuity of their condition, and using the results to efficiently allocate resources, including but not limited to hospital beds, staff, equipment, or supplies, and to avoid using invasive and potentially dangerous treatments, including, but not limited to, intubation and mechanical ventilation. This disclosure enables a method of screening subjects showing symptoms of an infection, for example a COVID-19 infection, and using the results to efficiently allocate test kits, for example real time RT-PCR test kits, to those most likely to test positive, and to efficiently allocate resources, including, but not limited to, hospital beds, staff, equipment, or supplies, when more direct tests, for example real time RT-PCR test kits, are not available.

This disclosure enables a system for collecting exhaled breath for use in methods of analyzing the exhaled breath for the presence of biomarkers associated with the pathogenesis of a disease, preferably a pandemic disease. This disclosure enables a system for collecting exhaled breath in such a way that bacteria or viruses are not present in the collected breath sample. This disclosure enables to collect breath samples of patients who have tested positive for COVID-19 and analyze the samples This disclosure enables a system for collecting exhaled breath comprising an air filter which removes pathogens including, but not limited to, bacteria and viruses, such as the COVID-19 corona virus.

This disclosure enables a two-stage system for collecting exhaled breath, wherein the first stage collects the air containing a pathogen including, but not limited to, bacteria, or viruses, further wherein the second stage collects exhaled breath with the pathogen removed, and in certain embodiments wherein the contents of the first stage are tested for the existence of the pathogen, in order to diagnose a condition including, but not limited to, a viral infection or a bacterial infection This disclosure enables a two stage system, wherein exhaled air containing a pathogen is captured in a first stage, and the first stage is subsequently placed in contact with a second stage, further wherein the air is forced into the second stage through a filter that removes the pathogen. This disclosure enables a breath collection device with a first stage and a second stage, wherein after the breath is passed from the first stage to the second stage, the first stage is decontaminated and/or disposed of.

This disclosure enables to collect breath in a device that is comprised of a sensor for one or more biomarkers of the severity of a disease state, wherein the test can be administered at the point of care, for example a drive through testing site, emergency room, or hospital bed, where the device returns the result of the test in real time without further transport or preparation of the sample, facilitating triage of patients This disclosure enables for the test to be conducted at the same site as the analysis is conducted, wherein the means of introducing the exhaled biomarkers into the analytical system is selected from the list including, but not limited to, capturing the breath and introducing the captured breath, in certain embodiments essentially immediately, into the analytical system, and having the subject exhale directly into the analytical system This disclosure enables a means (e.g., a collection kit) for collecting a breath sample remotely, for example in a home, work, or drive-through setting, and transporting the captured breath sample to a laboratory location for analysis.

This disclosure is advantageous in that a disease, including, but not limited to, a viral infection, a bacterial infection, or COVID-19 (including various variants disclosed herein), can be diagnosed rapidly at low cost. This disclosure is advantageous in that exhaled breath that that potentially contains pathogen can be captured for diagnosis of a disease state without contamination and potential exposure to pathogen of staff and equipment subsequent to the capture of the breath. This disclosure is advantageous in that captured breath can be used to determine the severity of the pathogenesis and/or pathophysiology disease state, including, but not limited to, active and ongoing formation of lung scar tissue or progressing pulmonary fibrosis, in order to triage patients and most efficiently use limited resources for the diagnosis and treatment of the disease state.

This disclosure is advantageous in that captured breath can be used to determine the severity of the pathogenesis and/or pathophysiology disease state, including, but not limited to, progressing pulmonary fibrosis, in order to triage patients and limit exposure to medical personnel to only those most in need of treatment, thereby minimizing infection to medical personnel and maximizing availability of medical personnel. This disclosure is advantageous in that breath biomarkers and exhaled pathogens may be collected in a single non-invasive collection event, allowing both indirect assessment of pathophysiology and preferably only if indicated by the indirect assessment, a direct testing of infection, thereby limiting invasive nasopharyngeal or blood sampling of very sick patients, and limiting exposure of medical personnel, facilities, and equipment to the pathogens.

This disclosure is advantageous in that the results of the test are returned by a device or system at the point of care quickly thereby assisting or enabling on the spot decisions as to, for example, whether the patients should be sent home, admitted to a standard hospital bed, or admitted to an ICU. This disclosure is advantageous in that the results of the test are returned by a device or system at the point of care, thereby preventing the contamination of equipment, facilities, and personnel that can occur with a test that requires transport and/or downstream processing.

These and other advantages, and features of this disclosure will become apparent to those persons skilled in the art upon reading the details of the formulations and methodology as more fully described below.

This disclosure enables a method and system of triaging patients during a pandemic. During a pandemic, treatment and medication will be in short supply. Pandemics are generally viral or bacterial infections. Often, they are associated with a severe lung infection. One recent example is a corona virus, such as COVID-19 (e.g., Wuhan variant, UK variant, South Africa variant, NYC variant).

Severe lung infections can have long term implications, for example scarring and pulmonary fibrosis, and it is believed that the active and ongoing formation of lung scar tissue, the rate of fibrosis formation, is a measure of the severity of the lung infection, and predictive of the need for intensive interventions such as admittance to an ICU and mechanical ventilation. The rate of progression of pulmonary fibrosis is impossible to determine with a single chest x-ray or CAT scan.

Some certain amino acids, specifically proline, 4-hydroxyproline, alanine, valine, leucine/isoleucine and allysine, are present in higher than normal levels during progressing pulmonary fibrosis and active and ongoing formation of lung scar tissue. In certain aspects, the levels of organic compounds in the breath are measured, and these levels are used to predict the future severity of the disease. Based on these results, the patient can be sent home, admitted to a regular hospital bed, or admitted to the ICU. An example of this type of test is the Diagnose Early COVID Acuity (DECA) test. Amino acids cannot readily be analyzed, without derivatization, by GC×GC-MS, which is why using high efficiency ionization Super-Secondary Electrospray Ionization (Super-SESI) apparatus (Fossil Ion Technology, Madrid Spain) coupled with high resolution mass spectrometry (HRMS) is a generally preferred route of analysis for the DECA test (or other suitable devices can be used). However, in certain embodiments a DECA test using GC×GC-MS that requires sample processing it utilized. In the Super-SESI device, the sample is introduced with the sample ideally at about 100% relative humidity at about 37° C. when entering the sample transfer line of the Super-SESI. There are various sample situations for DECA analysis using the Super-SESI ionization method, for example real-time breath collection as the patient provides one or several breaths directly into the Super-SESI device, or the breath is captured in a chamber or stainless steel bag (or another suitable bag whether including or excluding stainless steel). The breath can be captured as an exhaled breath condensate by a patient breathing into a chamber kept at about −30° C. to about −70° C. or liquid nitrogen temperature, about −196° C., for example, as disclosed in other Diagnose Early patent applications. The breath sample is reconstituted into a volume air/nitrogen while ensuring that the sample is at or close to about 100% relative humidity before introduction into the Super-SESI transfer line. Additionally, the breath can be captured using a breath chamber and concentrated in a breath cartridge submerged in liquid nitrogen and later reconstituted into a volume air/nitrogen while ensuring that the sample is at or close to about 100% relative humidity before introduction into the Super-SESI transfer line.

In certain aspects, an exhaled breath sample is captured in a breath chamber, and the levels of the relevant biomarkers in the sample are analyzed. The analysis can be done by sending the sample to a laboratory. However, there are issues related to doing this, including contamination with the pathogen and time delays. Thus, in certain embodiments a real-time point of care (rt-POC) system us used that captures a breath sample, and in real time (e.g., less than about 70, 60, 50, 40, 30, 20, 15, 10, 5 minutes) returns the results of the test. Sometimes, the test will be run on patients who have already tested positive for infection. However, pandemics are usually caused by novel pathogens, such as never before seen viruses, and the tests may be in very short supply, or they may take a long time to return results. In this case, the breath test may be performed, and the patients triaged based on the results, before the results of the test for infection with the pandemic pathogen are known. In cases of extreme shortage of the tests, the breath test may be used to determine who receives the pathogen test, or the pathogen test may be skipped entirely.

In certain aspects, some methods may comprise, but are not limited to, capturing a breath sample using a filter capable of capturing viruses and bacteria to protect others, measuring the relative quantities of certain multiple amino acids found in exhaled breath, using the measured quantities to diagnose a disease state, and preferably the acuity of a disease state, safely sending low acuity patients home, and focusing limited resources on providing life-saving treatment for high acuity patients.

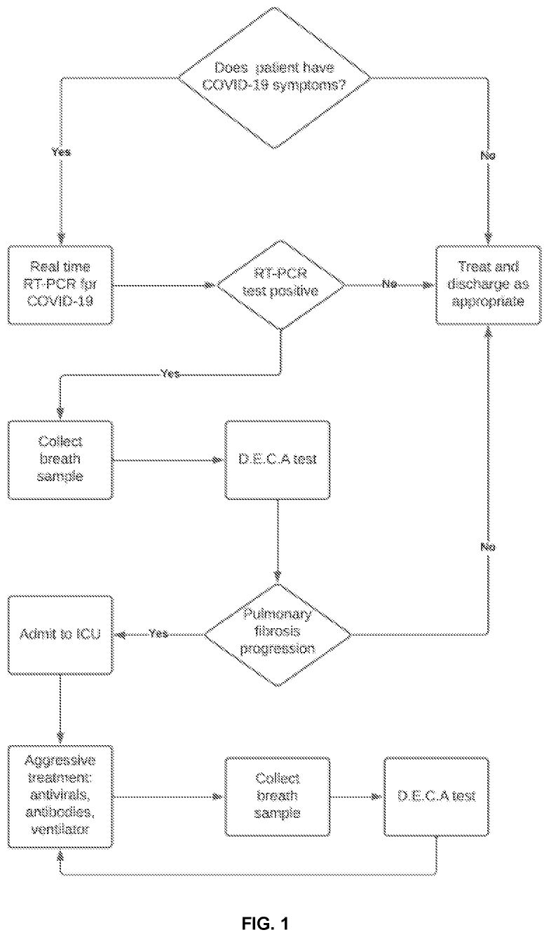

FIG. 1 shows one embodiment of the present disclosure, wherein a direct test for the condition is readily available and relatively fast. Preferably, the condition is a pandemic and it is projected, or already the case, that enough patients will require intensive treatment and/or medication that the treatment and/or medication will be in short supply. Preferably the condition is a virus or bacterial infection that can cause a severe lung infection. In one preferred embodiment, the pandemic pathogen is COVID-19 (e.g., Wuhan variant, UK variant, South Africa variant, NYC variant), and the test is real-time RT-PCR. One embodiment using the example of COVID-19 is shown in the flow chart of FIG. 1. In this embodiment, the test is readily available and the results are returned in two days or less, preferably in one day or less, more preferably the test results are available the same day.

If the patient tests negative for the pandemic pathogen, then the patient is treated as is usual for their condition. If the patient tests positive for the pandemic pathogen, then it is important to determine if they are in the sub-population whose survival will require intensive treatment, including, but not limited to, intubation and mechanical ventilation. As these intensive treatments are likely to be in very short supply, it is important that they be reserved for those patients whose survival will depend on them.

As described above, one potential consequence of lung infection that can have associated morbidity and mortality and is believed to be a predictor of acuity and the need for intensive treatment is active and ongoing formation of lung scar tissue and rapidly progressing pulmonary fibrosis. Pulmonary fibrosis can be diagnosed through chest x-rays or CAT scans, but it is unknown from a CAT scan or chest x-ray if the condition is preexisting or is caused or worsened by the pandemic pathogen infection.

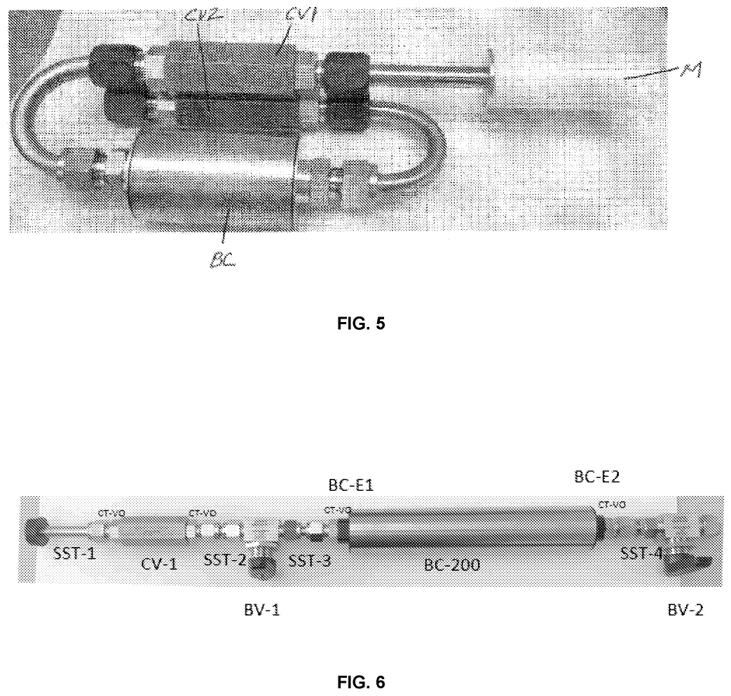

Because there are exhaled biomarkers for rapidly progressing pulmonary, and it is believed that the most acute pulmonary cases of pandemic pathogen infection will have rapidly progressing pulmonary fibrosis, i.e., active and ongoing formation of lung scar tissue, breath is collected from the patient who has tested positive for the pandemic pathogen using a breath chamber as shown in FIG. 1. Examples of embodiments the breath chamber, and the methods and systems for concentrating and assaying the breath for diagnostic biomarkers may include, but are not limited to, those shown in FIG. 5 and FIG. 6. FIG. 5 shows a 200 ml breath chamber (BC) with a check valve (CV1 and CV2) on either side and a mouthpiece (M), while FIG. 6 shows a breath chamber (BC-200) with ball valves (BV1 and BV2) on either side. Also shown in FIG. 6 are breath chamber end caps (BC-E1 and BC-E2), a check valve (CV-1), straight connectors with Viton O-ring (CT-VO) and stainless steel tubing (SST1-4).

It is important that the pathogens contained in the exhaled breath that is sampled are not passed on to later stages of the analysis process, to avoid contamination of equipment and personnel. This can be accomplished by filtering viruses and bacteria out of the exhaled breath sample. Preferred filters remove about 95% or more of the pathogen (e.g., 95, 96, 97, 98, 99), more preferably about 99% or more, more preferably about 99.9% or more. Preferred filters have a pore size of about 0.3 μm or less. Because corona viruses, like COVID-19 and SARS, have diameters of approximately 100 nm, then pore sizes of about 0.1 μm or less may be required. In one embodiment, pathogens are filtered out of the exhaled breath sample prior to introduction into the breath chamber to avoid contamination of the breath chamber and downstream processes. However, due to the required pore size and the concomitant high pressure drop of the filter and the potentially impaired lung function of the subject, it may be preferred to capture the breath by having the subject exhale into the breath chamber, then force the gas out of the chamber and through the filter using pressured gas, a piston, or any other means of pressurizing the breath sample in the breath chamber. This decontaminated breath sample may then be captured in a second chamber. Preferably, the captured breath sample is driven out of the breath chamber, through a filter, and into a concentrating system as described in FIG. 1. This breath condensate can then be warmed as required and injected into an analytical system, such as but not limited to a secondary electrospray MS system.

In certain embodiments, the exhaled breath capture and test is performed using a real time point of care (rt-POC) system comprised of a sensor for one or more compounds that are biomarkers of a condition that indicates lung infection acuity, for example progressing pulmonary fibrosis as a biomarker of infection with COVID-19 that is likely to require aggressive intervention, for example with a ventilator. In one embodiment, the rt-POC biomarker detection system requires the addition of other components of the analysis, for example reagents, before or essentially immediately after the breath sample is taken (e.g., within about 10, 9, 8 7, 6, 5, 4, 3, 2, 1 minutes). Preferably, the rt-POC system is supplied to the testing site with all required reagents prepackaged in or with the system. More preferably, the sensor requires no additional reagents. Preferred sensors are sensors that detect biomarkers, preferably volatile organic compounds, more preferably amino acids which may include one or more of but are not limited to proline, 4-hydroxyproline, alanine, valine, leucine/isoleucine and allysine.

Preferred rt-POC systems return test results in less than or about 10 hours, less than or about 5 hours, less than or about 1 hour, less than or about 30 minutes, less than or about 15 minutes, less than about 10 minutes, less than or about 5 minutes, or less than or about 1 minute.

Any detector that is capable of detecting VOCs, preferably amino acids, preferably one or more of proline, 4-hydroxyproline, alanine, valine, leucine/isoleucine and allysine, may be used, including currently available detectors and detectors to be developed in the future. Examples of detectors that might be used include, but are not limited to, immunosensors including but not limited to capacitive immunosensors, electrochemical immunosensors, optical immunosensors, microgravimetric immunosensors, thermometric immunosensors, antibody immunosensors, aptamer immunosensors, microRNA immunosensors; meso-tetra (4-sulphonatophenyl) porphyrin (TPPS) film sensors, colorimetric sensors, photochromic sensor chips, enzyme biosensors, organic electrochemical transistors, fluorescence sensors, electrochemiluminescence sensors, D-phenylalanine fluorescence sensors, enantioselective sensors, potentiometric sensors, ODAP-selective sensors, electrochemical metal ion sensors, microcantilever sensors, and supramolecular luminescent sensors.

In the rt-POC embodiment, the test is administered at the point of care, including, but not limited to, a drive-through testing site, a free-standing clinic, an emergency room, a hospital bed, and outdoor triage site, a temporary triage building such as a tent, or any other site or physically defined area that is used, built, or repurposed for diagnosis and or assignment of degree of urgency.

In certain embodiments, the rt-POC system may comprise a durable (i.e., multi-patient use) component that is removably attached to the breath chamber when the breath is captured. This durable component may comprise one or more of a system for measuring patient exhalation flow rate through the breath chamber, patient exhaled volume through the flow chamber, a system for informing the patient they are exhaling properly through the breath chamber, a system for indicating that the patient has exhaled a required amount through the breath chamber, a handle for holding the breath chamber, a mount for mounting the breath chamber, or an attachment to a base station.

In certain embodiments, the rt-POC system may comprise a base station to which one or more of the breath chamber, the durable component, and the sensor are docked after the breath is captured. After docking to the base station, the test is completed, and the results of the test are displayed and/or transmitted.

In certain embodiments, the rt-POC system comprises a system for entering information (e.g., a physical or virtual keyboard, a microphone, a camera), which may include, but is not limited to, patient name, patient date of birth, a patient identifying number or alpha-numeric string, the identity of the person giving the test, the location of the test, date, time, and the like. The system for entering the data may be part of the breath chamber, a base station, or it may be a paper document that documents the test, or a separate system, such as a computer workstation that allows entry of the data into an electronic patient record. The system for entering the data may also comprise a reader for identifying such things as durable systems, disposable components, reagents, patients, or caregivers. Readers may include but are not limited to: a bar code reader; a quick response (QR) code reader; a camera; an RFID reader; or a biometric system including, but not limited to, a finger print identifier, an iris recognition system, or facial recognition.

The rt-POC biomarker detection system may be comprised of one or more of: a breath chamber, a durable component, and a base unit, each of which may comprise one or more of: one or more biomarker detectors; one or more reagents; power for running the analysis; pumps, valves, reaction chambers, tubing and other fluidic or micro-fluidic components for concentrating the sample, moving (e.g., pushing, pulling, lifting, lowering) the sample, introducing reagents, and the like; one or more filters for removing contaminants such as viruses, a display for showing the status of the test, a display for showing the results of the test, a wired or wireless or waveguide system for transmitting data, including, but not limited to, patient data, time, date, location of the test, result of the test. The breath chamber or the durable component may comprise a system comprising a flow rate sensor, a flow volume sensor, or a pressure sensor, a system for calculating an exhalation flow rate; a system for calculating an exhaled volume, and a display, speaker, or other component for communicating information related to exhaled flow rate and/or exhaled volume. For example, some of such displays can include LCD, electrophoretic, plasma, digital, analog, or others. For example, some of such transmissions can be wired or wireless or via waveguide with suitable transmitters, receivers, transceivers, or other networking equipment.

In certain embodiments, the rt-POC system comprises a system for delivering information (e.g., a display, a speaker), which may include, but is not limited to, patient name, patient date of birth, a patient identifying number or alpha-numeric string, the identity of the person giving the test, the location of the test, date, time, the results of the test, a suggestion for future treatment, and the like. The system for entering the data may be part of the breath chamber, a base station, a paper document that documents the test, or a separate system, such as a computer workstation that allows entry of the data into an electronic patient record. If the breath sample is shown to have elevated levels of the relevant biomarkers, for example, but not limited to, biomarkers for rapidly progressing pulmonary fibrosis or active and ongoing formation of lung scar tissue, then the patient can be given intensive therapy, including therapies selected from a list which includes, but is not limited to: admission to an intensive care unit, treatment with pharmaceuticals including but not limited to anti-virals or anti-bacterials that may be in short supply, and intubation and mechanical ventilation.

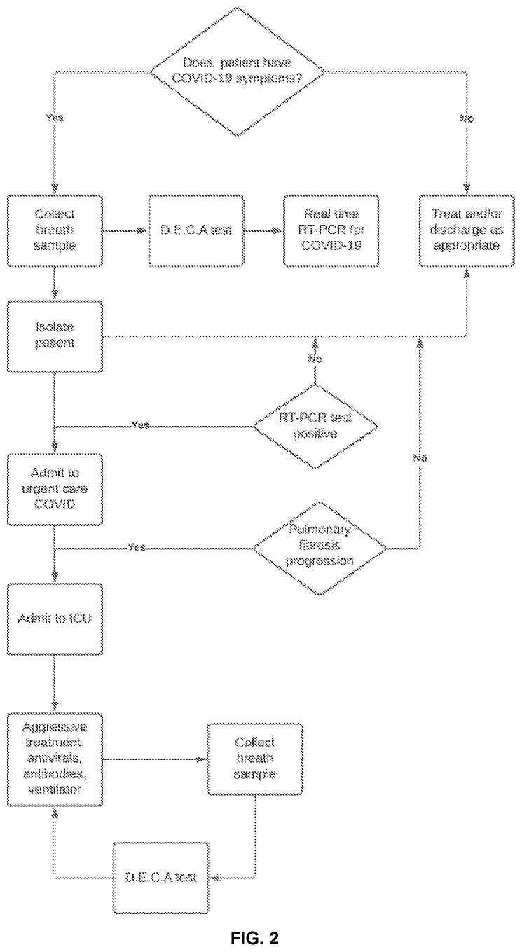

FIG. 2 shows another embodiment of the diagnosis and treatment method, which may be preferred when the test for the infection may be in short supply and/or take a significant time to return result. By way of example, as of 25 Mar. 2020, there were over 21,000 deaths associated with COVID-19, but there were not enough tests available to test all patients suspected of being infected with COVID-19. This can be seen from the CDC testing guideline at that time, which called for testing based on the following priorities (Table 1):

TABLE 1

| | |
|---|---|
| Priority 1 | Ensure optimal care options for all hospitalized patients, |
| | lessen the risk of nosocomial infections, and maintain the |
| | integrity of the healthcare system. |
| | Hospitalized patients and symptomatic healthcare workers. |
| Priority 2 | Ensure that those who are at highest risk of complication |
| | of infection are rapidly identified and appropriately triaged. |
| | Patients in long-term care facilities with symptoms, patients |
| | 65 years of age and older with symptoms, patients with |
| | underlying conditions with symptoms, and first responders |
| | with symptoms. |
| Priority 3 | As resources allow, test individuals in the surrounding |
| | community of rapidly increasing hospital cases to decrease |
| | community spread, and ensure health of essential workers. |
| | Critical infrastructure workers with symptoms, individuals |
| | who do not meet any of the above categories with |
| | symptoms, health care workers and first responders, and |
| | individuals with mild symptoms in communities |
| | experiencing high COVID-19 hospitalizations. |
| Non-Priority | Individuals without symptoms |

By way of example, if a patient has symptoms but is not yet critical and is not a healthcare worker, first responder, or a member of an at-risk population, they were only to be tested “as resources allow.” It would have been preferable to test anyone of priority 2 or below for biomarkers of severe progression of the disease and based on the results make a decision as to whether to test them for COVID-19 infection.

Also, as of March 2020, even those who were allowed access to the test had to wait a significant period, often as long as 10 days to receive the results. A way of triaging patients while waiting for the test results would clearly have resulted in better outcomes.

As can be seen in FIG. 2, another embodiment of the method appropriate when the test is in short supply would be to would be to first test for biomarkers using the method outlined in FIG. 1, and then if the biomarkers indicate expected disease acuity, testing for the pandemic pathogen, and treating accordingly.

In the case where the test for the pandemic pathogen takes many days to return results, one may choose to begin intensive therapy based on the biomarker results before it is known if the patient is infected with the pandemic pathogen.

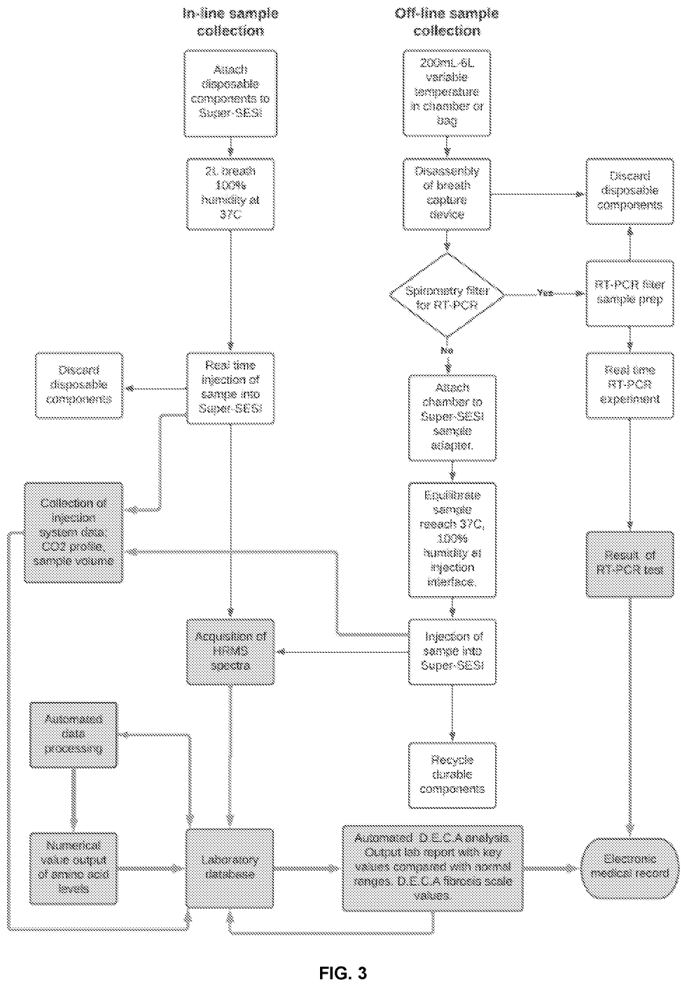

FIG. 3 details the DECA test procedures, separate from the clinical context, which details the DECA text box as shown in FIG. 1 and FIG. 2.

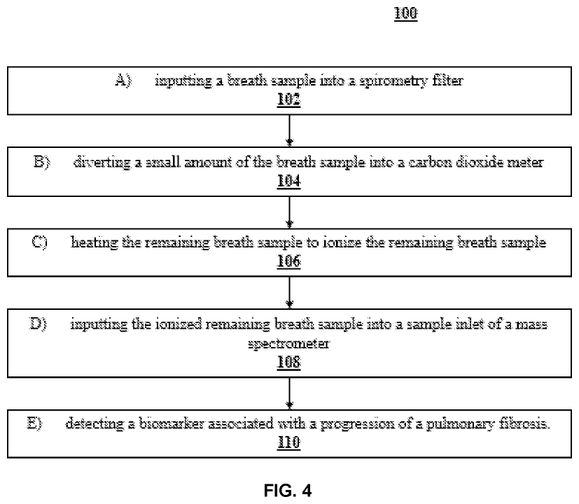

FIG. 4 details the physical steps 100 involved in one embodiment of testing a breath sample for a biomarker associated with a progression of a pulmonary fibrosis. A breath sample is input into a spirometry filter 102, while a small amount of the breath sample is diverted into a carbon dioxide meter 104. The remaining breath sample is heated to ionize the remaining breath sample 106, and the ionized breath sample is input into a sample inlet of a mass spectrometer 108. The output from the mass spectrometer detects a biomarker associated with or indicative of progression of pulmonary fibrosis 110.

In an alternative or additional embodiment, the presently disclosed DECA test could be used to follow recovery from lung damage cause by COVID-19 or other medical conditions. This could be natural recovery or recovery through medical treatment either conventional (by for example steroids) or emerging treatments by techniques such as stem cells or activation/reprogramming of cellular pathways by introduction of mRNA molecule or other genetic manipulation techniques. The rationale for this alternative use case is that the DECA test as outlined in this application is a measure of active collagen metabolism, both formation of fibers and degradation of fibers. Both processes releases proline, hydroxyproline and other amino acids when active; either during the establishment of fibrotic tissue by replacing damaged cells with fibers and during recovery through remodeling whereby fibrotic tissue is replaced by new cells derived from stem cells in the lung.

### EXAMPLES

The following examples are put forth so as to provide those of ordinary skill in the art with a complete disclosure and description of how to make and use the present disclosure, and are not intended to limit the scope of what the authors regard as their disclosure nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight or calculated exact molecular weight, temperature is in degrees Centigrade, and pressure is at or near atmospheric.

### Example 1

This example details the physical steps of real-time sample introduction into the Super-Secondary Electrospray Ionization (Super-SESI) apparatus (Fossil Ion Technology, Madrid Spain).

The Super-SESI apparatus, attached to the sample inlet of a mass spectrometer (for example Orbitrap CG-MS, Thermo Fisher Scientific, Waltham, Mass.), has a mouthpiece attached to a transfer line which leads the sample into the ionization chamber of the apparatus. A small amount of the sample is continuously diverted from the transfer line into a carbon dioxide meter (CO₂ meter) with a graphical interface (GUI). The mouthpiece could be a spirometry filter or pulmonary function testing filter or a spirometry mouthpiece without a filter or teflon tubing. In certain embodiments a spirometry filter with a virus filtration efficiency of about 99.99% and bacterial filtration efficiency of about 99.999% is used.

The patient or test subject exhales into the spirometry filter attached to the transfer line and is guided by the CO₂ meter GUI to maintain an even exhalation flow rate, for about 20-30 seconds until a total volume of at least about 2 L has passed through the apparatus. Alternatively, the breath sample about 50 mL to about 6 L collected offline can be introduced in a similar way into the Super-SESI apparatus can be captured and stored in a breath capture device. The transfer line would in this case have a tubing adapter attached instead of a spirometry mouthpiece. The breath collection device would be attached to the Super-SESI transfer line in a way which allows the optimal sample flow into the Super-SESI apparatus. The sample collection device could be heated up to about 30, about 70, about 100 or about 200° C. prior to sample transfer and maintained during sample transfer. Such a breath collection device could be a stainless steel sampling bag or a chamber made of stainless steel or other inert material such as a TEFLON® polymer.

The breath sample flowing through the ionization chamber of the apparatus is continuously ionized and analyzed by the attached high resolution mass spectrometer.

### Example 2

A clinical trial has been planned as follows:

Collect bedside sample using our breath collection device.

Blow into the device for one full breath.

Dispose of the anti-viral filter mouthpiece in the patient's room.

Wipe down the device using antiviral wipes outside the room per standard protocol (in an area that is clean).

Put breath collector in storage bag.

Transport to central lab at Stanford.

Test sample in central lab.

Aggregate clinical testing results.

Avoiding Contagion During Clinical Trials:

The anti-viral filters in mouthpiece keep the virus in the patient's room and eliminates spreading of the virus.

We follow existing hospital protocols for handling materials in patient's rooms who have COVID-19.

Physicians and hospitals workers will use the same PPE that they use today, no change in procedures.

### Example 3

The following is a sample draft grant application utilizing the current disclosure.

Measuring COVID-19 Severity.

DECA Test (Diagnose Early COVID Acuity Test).

Using Amino Acids to Assess Acuity of COVID-19 Progression.

Measuring the severity of a hospitalized patient's illness.

The Problem: 95% of COVID-19 patients will recover on their own with no drug therapy. 5% will become very ill.

The Solution: The present non-invasive test will help identify, at a very early stage, the patients at risk of becoming critically ill.

Background:

Our hypothesis is that our technology can be used to detect Rapid Growth of Lung Fibrosis (RGLF) as a leading indicator of COVID-19 severity. If so, we would be able to accurately predict if a patient will get well on their own; We would be able to accurately predict if a patient will require further treatment including hospitalization, mechanical ventilation, admittance to ICU, and advanced drug therapy.

Description:

Breath based VOC analysis to predict, early in COVID-19 infection, future need for mechanical ventilation using Secondary Electrospray Ionization (SESI), Gas Chromatography (GC), and High Resolution Mass Spectrometry (HRMS).

How the Biology Works:

Lung cells are anchored to basement membranes that are composed of a variety of proteins including collagen.

Collagen has the unique characteristic of having about 17% of its amino acid composition being the single amino acid proline, making collagen degradation identifiable in exhaled breath.

When Rapid Growth of Lung Fibrosis occurs, collagen breaks down and proline (among other amino acids) is detectable in exhaled breath in higher levels.

Our non-invasive technology can accurately detect these increased levels of specific amino acids, indicating the active growth of pulmonary fibrosis, that is a likely leading indicator for a patient's need for advanced treatment (hospitalization, ICU, mechanical ventilation, and drug therapy).

Competing Tests:

Existing technology cannot provide this type of screen.

Blood Tests: Blood has too many compounds, too complex, to generate an accurate signal. Breath analysis allows sampling of metabolic processes throughout the body. Breath collection is non-invasive, is painless, and can be sampled repeatedly. Breath contains approximately 2500 low molecular weight volatile compounds, and SESI coupled with Mass Spectroscopy can detect compounds that are present in very low abundance, in the parts-per-trillion (10e-12) to the parts-per-quintillion (10e-18) range. While similar metabolomic studies can be done with blood or headspace from blood, breath-based VOC analysis is more direct and provides easier sample handling and analysis. Breath VOC analysis can allow early detection and diagnosis of lung injury, and patient stratification for therapy selection and treatment monitoring, in a way that cannot currently be done using blood or imaging techniques.

CT scans and Chest x-rays cannot measure the rate of ongoing disease progression. These only measure the level of lung damage at the time of the test. There is “clinical radiological” dissociation, in that patients with COVID-19 can have continued rapid clinical progression in the absence of radiologic changes.

The Big Idea:

Simple amino acid biomarkers found in breath can accurately and non-invasively predict the clinical course of each patient and therefore guide treatment. Predicting which patients are going to advance to a life-threatening acute condition will allow hospitals to provide these patients with life-saving drug therapies and interventions such as mechanical intervention which may be in limited supply. Predicting which patients will recover without these therapies, including patients that will recover on their own and can be discharged to recover in the home setting will free up hospital beds, mechanical ventilation capacity, other equipment and supplies and physician's time, allowing these resources to be directed to those most in need.

Enabling Discovery and Approval of New Drug Therapies:

Today, pharmaceutical companies are testing new therapies on three types of patients: the 80% of patients who show mild or no symptoms, the 15% of patients that require some medical intervention but are not at risk for death, and the 5% of patients who require advanced care and drug treatment. Because the 80% will lead to very large placebo signals, achieving statistical significance may be difficult. By clinical designs wherein the acceptance criteria require our additional biomarker analysis for inclusion in the trial, only the 20% who require care, or the 5% who are at risk of death can be accepted into the trial, leading to higher statistical significance, enabling smaller clinical trial, and speeding identification and approval of life saving efficacious drugs.

How Our Test is Intended to Work in the Hospital Setting:

Patients who present with symptoms including cough, fever, and shortness of breath will be administered the real time RT-PCR test for infection with COVID-19.

We will administer our test to patients who test positive for COVID-19.

Our non-invasive breath test will be used to detect amino acid biomarkers, including proline, 4-hydroxyproline, alanine, valine, leucine/isoleucine and allysine and as well as a range of other biomarkers found in breath.

Based on our results and patient symptoms, COVID-19 positive patients will be triaged into three risk groups:

Life Threatening: Requiring the best available but potentially supply limited therapies.

Severe: Requiring hospitalization but will survive with more standard and readily available therapies. Will be monitored and moved to life threatening category as disease progression requires.

Mild/Moderate: Will recover well in a home setting.

How the Proposed Clinical Trials will work (clinical trial protocol overview):

Proposed study design:

The study design will be open label.

90 day longitudinal.

N=100 to 300 pending further analysis of statistical requirements.

Procedures:

Test for COVID-19 using real time RT-PCR test.

Conduct intake assessment of patients that test positive for COVID-19. Inclusion assessment will include assessment of symptoms (including cough, fever, shortness of breath) general state of health, questionnaire regarding known exposure to COVID-19, and informed consent.

Wearing personal protective equipment per hospital procedures, a technician, nurse, or doctor will collect breath sample in examination room or at bedside using Diagnose Early's breath collection device: Will instruct subject to:

Inhale fully.

Place mouthpiece in mouth and seal with lips.

Exhale for as long as is comfortable.

Dispose of the anti-viral filter and mouthpiece in the patient's room.

Wipe down the breath collection device using antiviral wipes in the anteroom per standard protocol.

Put breath collector in storage bag.

Transport to central lab.

Test sample in central lab.

Aggregate clinical testing results.

End Point:

Biomarker correlation with Covid-19 acuity.

Final clinical trial design, effect size, power calculation, statistical significance, etc. will be completed by the Stanford Quantitative Sciences Unit in collaboration with Diagnose Early technical team.

IRB approval will be on an accelerated schedule.

Contamination and Infection Control.

Will follow the standard and procedures of Stanford Medical Center for care of patients who are possibly infected with COVID-19:

Personal Protective Equipment (PPE).

Identifying, monitoring and reporting COVID-19 among hospitalized patients, volunteers, and staff (Hospital and Diagnose Early).

Additional safeguards to ensure no contamination from study procedures:

Biohazard disposal of mouthpiece and ant-viral filter in patient room.

Decontamination of breath collection device

Biohazard disposal of supplies used in device contamination.

Appropriate PPEs and written protocols to ensure safety of those involved in the collection and analysis of exhaled breath.

Technology Stack:

Breath collection device.

Non-invasive.

anti-viral filter.

Mouthpiece.

200 ml collection volume.

Sample prep procedures.

Secondary Electrospray Ionization.

High Resolution Mass Spectrometry.

### Example 4

This example describes a preferred DECA test report. The test report output would give a normalized numerical value for each amino acid part of the DECA test and/or a numerical value of the ratio of amino acid levels for amino acids that are part of the DECA test. The preferred scale would indicate normal value range in the overall population for all age group, for different age groups and other available cohort properties as they are available and confirmed by clinical studies. The preferred numerical value of the level for a certain amino acid in a person without lung damage is obtained as a result of a clinical trial using diverse cohorts which corresponds to large segments of the population. The preferred scale would indicate with which indicate ranges or values, with a numerical value, which indicate a probable low to moderate ongoing fibrosis process or high to severe ongoing fibrosis process.

### Example 5

This example describes a preferred process for obtaining the normalized value and/or ratios of amino acid levels in breath samples. The ionized compounds in the breath sample are analyzed in an HRMS instrument which outputs spectra at about 1 Hz. Sample introduction into the Super-SESI ionizer for about 20-30 seconds will generate about 20-30 spectra. Each spectrum results in about 2000-7000 ion species being measured. For each ion species mass (M/z) and instrument intensity is determined and recorded. In calibration experiments the mass (M/z) for the DECA amino acids have been determined. In calibration experiments the identity of ion species are determined.

The data from the HRMS instrument could be automatically verified to meet certain quality criteria which allow automated processing or could be verified manually by visual inspection of the output or a combination of manual or automated procedures. To process the data from the HRMS instrument, the instrument output data file, which can contain spectra from one breath sample or several breath samples, can be converted to the universal mzXML data format (or similar format) which allows interface with a range of processing programs which simplifies automated data processing: from raw data to the DECA test output value which represents the absolute or relative level of a certain amino acid in the breath sample.

The use of one mass spectroscopy processing program, or several programs used in sequence, could be automated using a scripting language which could be Python or similar scripting language. One preferred method to determine the level of an amino acid in breath is to integrate the intensity of a particular M/z (also termed extracted ion current, EIC) for a specific amino acid over the entire breath sample using intensities from about 20 to about 30 spectra. The integrated EIC can further be normalized to another compound ion unrelated to the DECA test and not affected by any disease or a compound, not found in breath, spiked into the breath sample prior to ionization.

The normalized value of the amino acid can further be normalized to the measured volume of the breath sample or the fractional volume of a breath sample which has a certain carbon dioxide concentration as measured during sample introduction into the Super-SESI-HRMS. The final normalized value for a certain amino acid level can be divided with the value of another amino acid value to create ratios between amino acid levels. Ratios can be used to either reduce the number of output numerical values or potentially indicate processes such as collagen production versus elastin production/degradation.

The total time using from end of data acquisition using the HRMS to final DECA test output could be less than about 1 second or less than about 5 second or less than about 10 seconds.

This disclosure is shown and described herein in a manner which is considered to be the most practical and preferred embodiments. It is recognized, however, that departures may be made therefrom which are within the scope of the disclosure and that obvious modifications will occur to one skilled in the art upon reading this disclosure.

While this disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

1. A method of allocating medical resources during a pandemic, the method comprising:
a) screening a multiplicity of patients based on known symptoms;
b) testing each of the patients for infection due to a pathogen;
c) capturing exhaled breath from the patients, wherein the pathogen is removed from the captured breath;
d) determining the levels of a compound in the exhaled breath of the patients; and
e) based on the determined levels, allocating medical resources to the patients.

2. The method of claim 1, wherein the pathogen is a virus is selected from a corona virus, an influenza virus, a syncytial virus, a parainfluenza virus, an adenovirus, a rhinovirus, a metapneumovirus and an enterovirus.

3. The method of claim 1, wherein the pathogen is a virus, and the compound is a biomarker for progression of pulmonary fibrosis.

4. The method of claim 3, wherein the virus is COVID-19.

5. The method of claim 4, wherein the testing comprises a real-time RT-PCR test.

6. The method of claim 3, wherein the medical resources are a hospital bed, a bed in an intensive care unit, a ventilator, an endotracheal tube, a doctor, a nurse, an x-ray, a CAT scan or an anti-viral medicine.

7. The method of claim 1, wherein the pathogen is selected from a virus and a bacteria, and wherein the pathogen is removed using a filter.

8. The method of claim 7, wherein the filter is selected from a glass fiber filter, a polycarbonate membrane filter, and an activated charcoal filter.

9. The method of claim 8, wherein the filter has an effective pore size of about 0.3 μm or less.

10. The method of claim 9, wherein the effective pore size is about 0.1 μm or less.

11. The method of claim 7, wherein the filter removes about 95% or more of the pathogen from the breath sample.

12. The method of claim 11, wherein the filter removes about 99% or more of the pathogen.

13. The method of claim 12, wherein the filter removes about 99.9% or more of the pathogen.

14. The method of claim 7, wherein the exhaled breath travels the filter prior to being captured.

15. The method of claim 7, wherein the exhaled breath is forced through the filter after being captured.

16. The method of claim 15, wherein the breath is captured in a breath capture system, and the breath capture system is disposed of after forcing the breath sample through the filter.

17. The method of claim 11, wherein the testing is done after the breath capture, and the pathogen that is captured by the filter is analyzed to diagnose the infection by the pathogen.

18. The method of claim 3, wherein the biomarker is proline, 4-hydroxyproline, alanine, valine, leucine/isoleucine, allysine or any combination thereof.

19. The method of claim 18, wherein the testing comprises measuring the level of proline, 4-hydroxyproline, alanine, valine, leucine/isoleucine, or allysine.

20. The method of claim 19, wherein the testing comprises measuring the ratio of the level of two or more or four or more of proline, 4-hydroxyproline, alanine, valine, leucine/isoleucine, or allysine.

21. The method of claim 20, wherein the testing comprises measuring the levels of proline, 4-hydroxyproline, alanine, valine, leucine/isoleucine, and allysine.

22. The method of claim 4, wherein the testing comprises a real-time point of care (rt-POC) test.

23. The method of claim 22, wherein the rt-POC test returns results in less than or about 1 hour.

24. The method of claim 23, wherein the rt-POC test returns results in less than or about 10 minutes.

25. The method of claim 24, wherein the rt-POC test returns results in less than or about 1 minute.

26. The method of claim 22, wherein the rt-POC test comprises a system comprised of:
a) a breath chamber;
b) a sensor; and
c) a system for displaying the result of the test; or
d) a system for transmitting the result of the test.

27. The method of claim 26, further comprising one or more of a durable component configured to attach to the breath chamber, a base unit, a biomarker detector, a reagent, a pump, a valve, a reaction chamber, a filter, a display, a flow rate sensor, a flow volume sensor, a pressure sensor, a system for calculating an exhalation flow rate, a system for calculating an exhaled volume, a display, or a speaker.

28. The method of claim 26, wherein the sensor is an electrochemical immunosensor, an optical immunosensor, a microgravimetric immunosensor, a thermometric immunosensor, an antibody immunosensor, an aptamer immunosensor, a microRNA immunosensor, meso-tetra (4-sulphonatophenyl) porphyrin (TPPS) immobilized onto a film, a colorimetric sensor, a photochromic sensor chip, an enzyme biosensor, an organic electrochemical transistors, a fluorescence sensor, an electrochemiluminescence sensor, a D-phenylalanine fluorescence sensor, an enantioselective sensor, a potentiometric sensor, an ODAP-selective sensor, an electrochemical metal ion sensor, a microcantilever sensor, or a supramolecular luminescent sensor.

29. A method for testing for a biomarker associated with progression of pulmonary fibrosis, the method comprising:
a) introducing a breath sample into a spirometry filter;
b) diverting a small amount of the sample into a carbon dioxide meter;
c) heating the remaining sample to ionize the sample;
d) introducing the ionized sample into the sample inlet of a mass spectrometer; and
e) detecting a biomarker associated with progression of pulmonary fibrosis.

30. The method of claim 29, wherein the biomarker is proline, 4-hydroxyproline, alanine, valine, leucine/isoleucine, allysine or any combination thereof.

31. A method of treating a patient for a viral or bacterial infection characterized by progression of pulmonary fibrosis, the method comprising:
a) introducing a breath sample from the patient into a spirometry filter;
b) ionizing the breath sample;
c) introducing the ionized sample into the sample inlet of a mass spectrometer;
d) detecting a biomarker associated with progression of pulmonary fibrosis; and
e) treating the patient with the detected biomarker.

32. The method of claim 31, wherein the patient has a viral infection.

33. The method of claim 32, wherein the viral infection is a corona virus, an influenza virus, a syncytial virus, a parainfluenza virus, an adenovirus, a rhinovirus, a metapneumovirus and an enterovirus infection.

34. The method of claim 31, wherein the biomarker is proline, 4-hydroxyproline, alanine, valine, leucine/isoleucine, allysine or any combination thereof.
